(19) United States
(12) Patent Application Publication
(10) Pub. No.: US 2022/0249036 A1
(43) Pub. Date: Aug. 11, 2022

(54) RADIATION SHIELDING DEVICES, SYSTEMS, AND METHODS

(71) Applicant: Radux Devices, LLC, Omaha, NE (US)

(72) Inventors: Andrew Ubel, St. Paul, MN (US); Kevin Jay Wiersma, Maple Grove, MN (US); Thomas Joseph Monette, Andover, MN (US)

(21) Appl. No.: 17/730,846

(22) Filed: Apr. 27, 2022

Related U.S. Application Data
(63) Continuation-in-part of application No. 17/085,594, filed on Oct. 30, 2020.

(51) Int. Cl. A61B 6/10; G21F 3/00

(52) U.S. Cl. CPC A61B 6/107; G21F 3/00

(57) ABSTRACT

In general, radiation shielding systems that shield radiation from multiple directions are described. In one embodiment, method of shielding radiation is provided, including orienting the rigid radiation shield in a selected position relative to a patient, and attaching a flexible radiation shield to the rigid radiation shield such that the flexible rigid radiation shield includes a first generally vertical portion that covers a gap between the rigid radiation shield and the patient.

### CROSS-REFERENCE TO RELATED APPLICATIONS

This application is a continuation-in-part of U.S. application Ser. No. 17/085,594, filed on Oct. 30, 2020, the contents of which are hereby incorporated by reference in their entirety.

### TECHNICAL FIELD

This document describes devices, systems and methods for shielding radiation in a medical environment, such as portable radiation shielding devices for use in shielding healthcare practitioners from radiation.

### BACKGROUND

Healthcare practitioners often work near a radiation field, such as from a fluoroscope, X-ray machine, or other imaging system, when treating a patient. Procedures and therapies are often designed to reduce patient exposure while allowing healthcare practitioners to effectively treat the patient. However, cumulative radiation exposure of physicians and healthcare practitioners may be significant as they often perform multiple treatments in a typical day, and radiation exposure may be increased when a particular treatment requires the healthcare practitioner's body to be close to a field of radiation. For example, the healthcare practitioner's hands may be exposed to radiation from fluoroscopic imaging equipment when inserting a catheter in a patient's vessel, or when delivering other instruments, medicines, fluids, or other endovascular devices in a patient's vessel. Various techniques have been used to limit radiation exposure, such as physical barriers including radiation shielding and bodywear.

### SUMMARY

Some embodiments described herein include devices, systems, and methods that can be used to provide protection for a healthcare practitioner, such as a physician, nurse, technician, etc., during a medical procedure. For example, a radiation shielding device is described that is positionable to shield radiation from multiple directions, such as by providing shielding in a generally horizontal orientation and in a generally vertical orientation. In some embodiments, the device is attachable to a structure to orient and/or support a first portion in a generally vertical orientation while a second portion is in a generally horizontal portion. In some embodiments, the radiation shielding device includes an attachment component that facilitates attachment with a complementary surface or feature of a support structure. For example, the radiation shielding device is attachable with a rigid shielding device (e.g., supported from a ceiling, table, equipment, etc.,) in the operating room to at least partially support and/or orient a first portion of the radiation shielding device while a second portion of the radiation shielding device is at least partially supported by a patient.

In an example embodiment, a radiation shield may be connected to an elongate neck, and the neck manipulable into a user-selectable position to shield the healthcare practitioner, while facilitating efficient workflow in an operating environment. The neck may have sufficient length to extend between a base or support location and a shield attachment location. The radiation shield may include a relatively rigid and/or shape-stable radiation shield, and/or may include a relatively flexible and/or non-shape-stable radiation shield (e.g., a radiation shielding drape). In some examples, the radiation shield may optionally be attached to or at least partially supported by a crossbar feature. The relatively flexible and/or non-shape-stable radiation shield may optionally have a length that is greater than at least 50% of the length of the elongate neck.

In some optional embodiments, the radiation shielding device may shield radiation from multiple directions, such as by providing shielding in a generally horizontal orientation and in a generally vertical orientation. For example, the radiation shield may include a crossbar that facilitates support of the radiation shield to provide both a horizontal radiation barrier and a vertical radiation barrier. The radiation shield may include a first portion that extends vertically (e.g., at least partially below the crossbar) and a second portion that extends in a direction different from the first portion, such as in a horizontal direction. The radiation shield may include a relatively flexible and/or non-shape-stable radiation shield attached to the crossbar. A first portion of the relatively flexible and/or non-shape-stable radiation shield may extend at least partially in a first direction (e.g., a substantially vertical orientation) and a second portion of the relatively flexible and/or non-shape-stable radiation shield may concurrently extend in a second direction different from the first direction (e.g., at least partially in a substantially horizontal orientation/orthogonal to the neck). Alternatively, or additionally, the radiation shield may include a relatively rigid and/or shape-stable radiation shield. The relatively rigid and/or shape-stable radiation shield may extend at least partially in a first direction (e.g., a substantially vertical orientation). The relatively rigid and/or shape-stable radiation shield may include a second portion that extends at least partially in a second direction different from the first direction (e.g., at least partially in a substantially horizontal orientation/orthogonal to the neck).

The radiation shielding device can protect the healthcare practitioner's hands, arms, and body that may otherwise be exposed to relatively higher levels of radiation. In addition, the radiation shielding device can protect the patient's body that may otherwise be exposed to relatively higher levels of radiation. In various example configurations, the radiation shield, and in some optional embodiments the base (or portions thereof), are fabricated from a radiation shielding material such that unsafe levels on first sides of the radiation shield and base (or portions) may be reduced to safe levels on second sides of the radiation shield and base (or portions).

In some optional embodiments, the radiation shielding device may include an elongate neck that may attach to the radiation shield at a first end and to the base at a second end. The elongate neck can be flexible and/or shape adjustable. In some optional embodiments, the elongate neck may be manipulated by a user to position the radiation shield in a selected orientation relative to the base during set-up in the operating environment. The elongate neck may be magnetically coupled to the base. Alternatively, or additionally, the base may comprise a magnetic material for magnetically attaching to a supporting surface. In some optional embodiments, the base may comprise a clamp to secure the radiation shielding device to a supporting object.

In some optional embodiments, the neck may be coupled to a support element (e.g., a base, surgical table, imaging equipment, patient, etc.) while a sterile drape is positioned between the neck and the support element. For example, the neck and support element may be magnetically coupled through the sterile drape while the sterile drape remains unbroken, promoting sterility of an area below the sterile drape.

In some optional embodiments, a base may be stabilized partially or entirely by an object (e.g., a patient) in close proximity to a radiation source and/or an imaging location. In some optional embodiments, the base may be radio-transparent. Alternatively or in addition, the base may include portions that are radio-opaque to at least partially block radiation (e.g., such that unsafe levels on a first side of the base (or portions) may be reduced to safe levels on a second side of the base (or portions)).

Particular embodiments described herein provide a radiation shielding device, including a radiation shield, an elongated neck having a first end and a second end, the elongated neck configured to attach to the radiation shield at the first end, and a base including a structure for engaging the second end of the elongate neck.

In some implementations, the device may optionally include one or more of the following features. The radiation shield that may be flexible. A first portion of the radiation shield may be configured to rest on a patient and a second portion of the radiation shield may be configured to be supportable by the elongate neck. The radiation shield may define an opening defined through a thickness of the radiation shield, the slit configured to allow passage of an interventional tool from a first side of the first portion of the radiation shield to a second side of the first portion of the radiation shield. The radiation shield may be rigid. The radiation shield may include barium sulfate. The length of the radiation shield may be more than at least 50% than the length of the elongate neck. The first end of the elongate neck may include a crossbar attachable to the radiation shield. The radiation shield may extend from the crossbar in a plane that is transverse to a width of the base. At least a portion of the elongate neck may be flexible. The elongate neck may be a gooseneck, a flexible tube, or a flexible pipe. The second end of the elongate neck may include a magnet configured to magnetically attach to the structure of the base. The second end of the elongate neck may include a coupling configured to attach to the structure of the base. The base may have a flat surface configured to be positioned underneath a patient. The base may include a clamp to secure the radiation shielding device to an object. The clamp may be a pinch clamp. The clamp may be a screw clamp. The base may include a magnetic material for magnetically attaching the base to a supporting object. The magnetic material may include one or more magnets disposed on a surface of the base. The device may further include a magnetic disk configured to magnetically couple the base to the supporting object. The base may be radio-transparent.

Particular embodiments described herein provide a method of shielding radiation, including positioning a base of a shielding device under the weight of an object proximate a radiation source, attaching a first end of an elongate neck to a radiation shield, attaching a second end of the elongate neck to the base, and flexing the elongate neck to adjust a position of the radiation shield such that a first portion of the radiation shield is supported by the elongate neck and a second portion of the radiation shield is supported by an object.

In some implementations, the method may optionally include one or more of the following features. In some examples, the object may be a patient. Positioning the base may include sliding the base underneath a patient.

Particular embodiments described herein provide a method of shielding radiation, including supporting a shielding device at least partially by a patient, attaching a shielding device to an object to at least partially support a first portion of the shielding device in a first orientation while a second portion of the shielding device is in a second orientation different from the first orientation.

Particular embodiments described herein provide a method of shielding radiation, including covering a rigid radiation shield with a plastic cover, orienting the rigid radiation shield in a selected position relative to a patient, attaching a flexible radiation shield to the rigid radiation shield such that the flexible rigid radiation shield includes a first generally vertical portion that covers a gap between the rigid radiation shield and the patient. The plastic cover may be located between the rigid radiation shield and the flexible radiation shield while the flexible radiation shield is at least partially supported by the rigid radiation shield.

In some implementations, the method may optionally include one or more of the following features. The flexible radiation shield includes a second portion oriented transverse to the first portion while the first portion is attached to the rigid radiation shield. The second portion is at least partially supported by the patient while the first portion is at least partially supported by the rigid radiation shield. The method may include positioning the flexible radiation shield relative to the patient. Positioning the flexible radiation shield includes folding the first portion relative to the second portion. Attaching the flexible radiation shield includes attaching by an attachment component of the flexible radiation shield. The attachment component includes a magnetic component. The magnetic component is located entirely internally within the flexible radiation shield. The radiation shield is flexible. The radiation shield includes a predetermined fold location, and positioning the flexible radiation shield includes folding the flexible radiation shield about the predetermined fold location. The radiation shield includes a radiation shielding sheet. The attachment component of the flexible radiation shield includes a clamp. The base comprises a magnetic material configured to magnetically attach the base to an object. The method may include attaching a magnet to the rigid radiation shield, and attaching the flexible radiation shield includes bringing an attachment component of the flexible radiation shield into magnetic engagement with the magnet. The magnet is adhesively attached to the rigid radiation shield.

Particular embodiments described herein provide a radiation shielding device, including a first flexible portion comprising a radiation shielding material, a second flexible portion comprising a radiation shielding material, and a magnetic attachment component located internally within the first flexible portion, the magnetic attachment component configured to magnetically engage a magnetic object to secure the first flexible portion in a selected position relative to the second flexible portion.

In some implementations, the radiation shielding device may optionally include one or more of the following features. The radiation shield includes a fold location between the first flexible portion and the second flexible portion. The first flexible portion is positionable in a generally vertical position when secured by the attachment component while the second flexible portion is positioned in a generally horizontal position. The first flexible portion is at least partially supported by attachment of the attachment component to an object in an operating environment while the second flexible portion is at least partially supported by a patient. The magnetic attachment component is configured to secure the radiation shielding device over a plastic cover without breaking the plastic cover.

Some embodiments of the devices, systems and techniques described herein may provide one or more of the following advantages. First, some embodiments described herein may reduce the level of radiation a healthcare practitioner may be exposed to. For example, an example radiation shielding device can provide radiation shielding in multiple directions (e.g., generally vertical and horizontal directions). Moreover, the radiation shielding device may provide a high level of protection from both direct radiation and scatter radiation directed towards the healthcare practitioner from a range of directions. Furthermore, the radiation shielding device may provide a high level of concurrent protection (e.g., attenuation) to the healthcare practitioner and the patient (e.g., portions of the patient's body spaced from a target area). In some embodiments, the radiation shielding device can provide a level of attenuation of more than 50%.

Second, some embodiments described herein help maintain or improve sterility within the sterile field. For example, radiation shielding devices may include a sufficiently strong magnetic attachment structure that enables a healthcare practitioner to couple the base to the elongate neck while the thickness of one or more surgical drapes is between the base and neck. In some examples, the radiation shielding devices may include a base including a clamp that can clamp over one or more surgical drapes (e.g., without breaking the surgical drape). In some examples, the radiation shielding devices may include a magnetic base that can magnetically attach to an equipment surface covered by one or more surgical drapes. Potentially non-sterile surfaces are maintained separate from the sterile field during operation and/or positioning of the radiation shielding device during or after set-up of the medical location.

Third, some embodiments described herein may facilitate precise positioning of a radiation shield proximate a target area of radiation delivery. For example, in some optional embodiments, the elongate neck is a flexible or shape-adjusting neck that facilitates positioning of the radiation shield in a user-selected position (e.g., the angle, curvature, orientation, etc. may be adjusted to any angle). For example, an elongate neck and crossbar attachable to the radiation shield facilitates positioning of the radiation shield and/or imparting the radiation shield with a desired shape or curvature (e.g., a portion of the flexible crossbar may be bent such that the radiation shield is positioned in a curved configuration).

Fourth, the base and radiation shield can be configured to avoid interference between a radiation source and target area. In various example embodiments, the base may be radio-transparent and/or include a cut-out region (e.g., provided by a “U”-shaped base.

Fifth, some embodiments described herein provide a high degree of radiation shielding while facilitating efficient operation by the healthcare practitioner. The radiation shielding device may be positioned in a user-selected location and/or a user-selected orientation. The flexibility in positioning and orienting the radiation shielding device allows the healthcare practitioner to position the device relative to the healthcare practitioner's preferred operating position. In some embodiments, the radiation shielding device may be partially or entirely malleable such that the healthcare practitioner may bend the radiation shielding device into a selected configuration, and the radiation shielding device retains the selected configuration during operation. Alternatively, or additionally, the orientation of one or more radiation shielding devices may further enhance efficient operation by the healthcare practitioner, such as by providing a relatively larger area of protection that facilitates free movement by the healthcare practitioner during a medical procedure while the healthcare practitioner remains in an area substantially shielded from radiation exposure. Furthermore, some embodiments provide flexibility in providing a shielding zone that is distant or otherwise spaced from a support location. For example, an elongate neck may extend between a support location, such as a support location at a height below the patient, to a radiation shielding zone at a height above a patient. The radiation shielding device may thus facilitate selection of a desired support location relatively more independent of the location of the radiation shielding zone.

Sixth, some embodiments described herein facilitate efficient set-up of the operating environment. For example, a radiation shielding device including a base including a magnet, a clamp, or an adhesive to removably attach to the elongate neck and/or a supporting object may reduce the time the healthcare practitioner spends setting up the operating environment. Furthermore, the healthcare practitioner may have increased flexibility to quickly adjust and/or remove or add additional radiation shielding devices during a medical procedure.

Seventh, some embodiments described herein facilitate efficient operation in the medical environment. For example, a radiation shielding device including a passage through the radiation shield may facilitate passage of a medical device (e.g., a tubular medical device, sheath, interventional tool, or other device) from a first side of the radiation shield (e.g., facing the healthcare practitioner) to a second side of the radiation shield (e.g., in the direct field of radiation below a radiation source). The healthcare practitioner may efficiently and effectively manipulate the device while the device has a direct path to a patient access point or other location within the direct field of radiation.

Eighth, some embodiments described herein facilitate flexible positioning of the radiation shielding device such that the healthcare practitioner can operate from a medically advantageous location of the patient. An operator may thus operate from a location selected primarily based on advantages in patient care while being less constrained by ergonomic or radiation dosage requirements, for example.

Ninth, some embodiments described herein provide a substantially continuous zone of protection by providing a continuous shield that includes portions in a vertical and/or horizontal orientation that are positioned at an angle relative to one another. For example, some example radiation shielding devices may include a substantially lower shielding portion and a substantially upper portion. The radiation shielding device preferably does not include an unshielded break or opening between the substantially lower shielding portion and substantially upper portion that might otherwise allow a direct path for radiation to pass between first and second sides of the radiation shielding device, and thereby can provide a substantially continuous zone of protection for the healthcare practitioner. Furthermore, the radiation shielding device can provide concurrent radiation protection to both the healthcare practitioner and the patient by creating vertical and horizontal barriers.

Tenth, some embodiments described herein provide modularity of radiation shielding device components that facilitate sterilization of the radiation shielding device. For example, the healthcare practitioner may sterilize the base, neck, and/or shield individually. In some embodiments, the neck/shield may be sterilized and reused independent of a base, or vice versa.

The details of one or more implementations are set forth in the accompanying drawings and the description below. Other features and advantages will be apparent from the description and drawings, and from the claims.

### DESCRIPTION OF DRAWINGS

The present description is further provided with reference to the appended Figures, wherein like structure is referred to by like numerals throughout the several views, and wherein:

FIG. 1 is a perspective view of an example radiation shielding device in a medical environment, including a flexible shield attached to a base via a malleable neck.

FIG. 2A is a perspective view of the example radiation shielding device of FIG. 1.

FIG. 2B is a partial exploded view of the example radiation shielding device of FIG. 1.

FIG. 3 is a perspective view of an example radiation shielding device in a medical environment, including a flexible shield attached to a base via a malleable neck.

FIG. 4 is a perspective view of an example radiation shielding device including a rigid shield attached to a base via a malleable neck.

FIG. 5 is a perspective view of an example radiation shielding device including a pinch clamp.

FIG. 6 is a perspective view of an example radiation shielding device including a screw clamp.

FIG. 7 is a perspective view of an example radiation shielding device including a base.

FIG. 8 is a side view of an example disk attached to radiation equipment.

FIGS. 9A and 9B are perspective views of an example radiation shielding device in a medical environment.

FIG. 9C is a perspective view of the example radiation shielding device shown in FIGS. 9A and 9B.

FIG. 10 is a flow diagram of an example method of shielding radiation in a medical environment.

### DETAILED DESCRIPTION OF ILLUSTRATIVE EMBODIMENTS

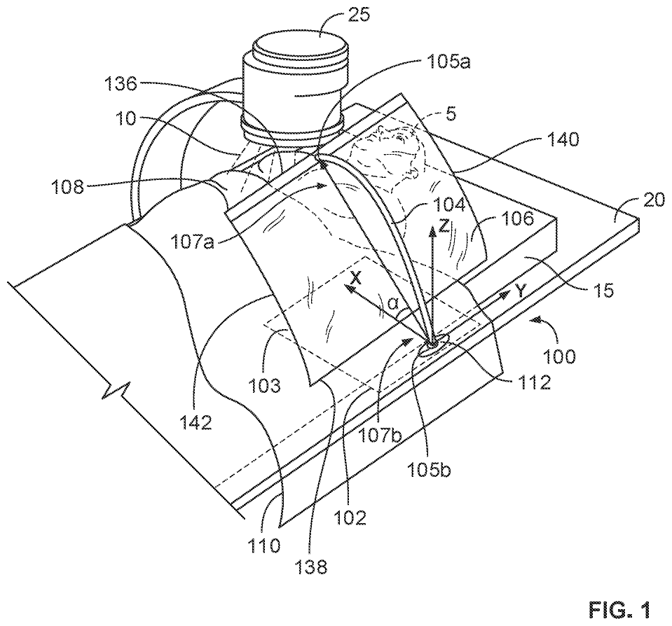

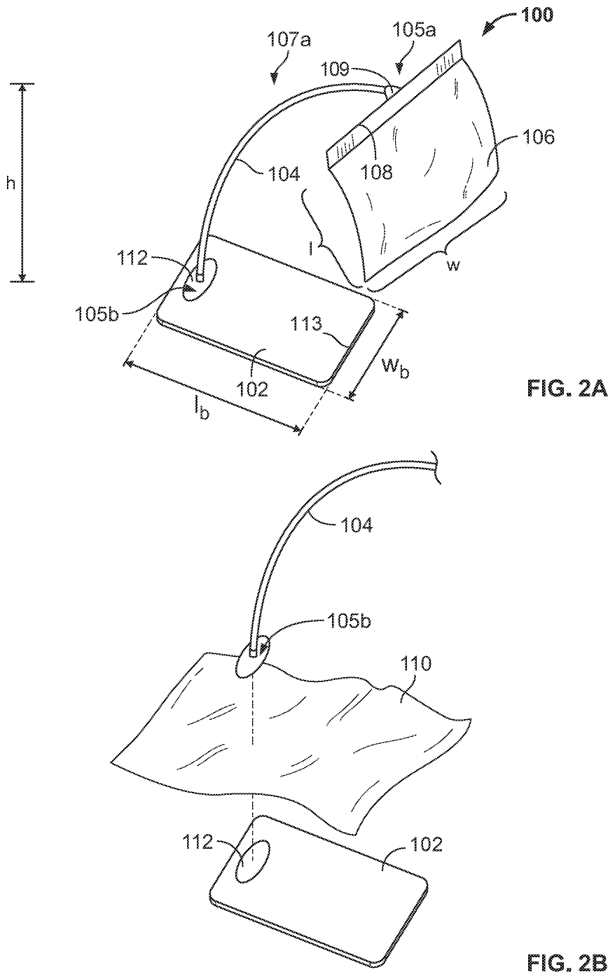

Referring to FIGS. 1-2, an example radiation shielding device 100 is shown, including a base 102, a neck 104, and a radiation shield 106. The radiation shielding device 100 is positioned near a patient 5 and a radiation source 25 (e.g., such as a medical imaging device) to shield a person (e.g., a healthcare practitioner) positioned near the radiation 10 emitted by the radiation source 25 during a medical procedure. For example, the radiation shielding device 100 can be positioned between the radiation source 25 and the healthcare practitioner. Radiation source 25 can include an emitter configured to emit radiation 10 and a detector configured to receive radiation 10 emitted by the emitter.

A user of radiation shielding device 100 (e.g., a healthcare practitioner) may arrange base 102, neck 104, and radiation shield 106 prior to an operation involving radiation source 25. Base 102 may be stabilized by an object, such as the body weight of patient 5 to be subjected to radiation 10 emitted by radiation source 25. In some example embodiments, base 102, neck 104, and radiation shield 106 are initially unconnected. Base 102 may be positionable underneath patient 5 (e.g., between mattress 15 and table 20) by the healthcare practitioner. The healthcare practitioner may subsequently attach neck 104 to base 102 and further attach the radiation shield 106 to neck 104. Alternatively, the healthcare practitioner may attach radiation shield 106 to neck 104 and subsequently attach neck 104 to base 102. In some embodiments, neck 104 or radiation shield 106 attached to neck 104 may be attached to base 102 before base 102 is positionable underneath an object (e.g., patient 5).

In some embodiments, neck 104 may include multiple neck portions that can be connected to form neck 104. For example, a first portion of neck 104 may be integrally connected with radiation shield 106 and a second portion of neck 104 may be integrally or reversibly attached to base 102. The healthcare practitioner may attach the first portion of neck 104 to the second portion of neck 104 and may subsequently attach the second portion of neck 104 to base 102. The healthcare practitioner may subsequently attach the second portion of neck 104 to base 102 before base 102 is positioned underneath an object (e.g., patient 5). Alternatively, the healthcare practitioner may subsequently attach the second portion of neck 104 to base 102 before base 102 is positioned underneath an object (e.g., patient 5). In some embodiments, the first portion of neck 104 that is integrally connected with radiation shield 106 is longer than the second portion of neck 104. In some embodiments, the first portion of neck 104 that is integrally connected with radiation shield 106 has a length that is more than 100%, more than 150%, more than 200%, more than 250%, or more than a length of the second portion of neck 104.

The healthcare practitioner can position radiation shield 106 in a user-selected position (e.g., by adjusting the angle, curvature, orientation, etc.) to provide a selected zone of radiation shielding for the healthcare practitioner while facilitating ergonomic and efficient operation. For example, neck 104, and/or crossbar 108 attachable to the radiation shield 106, facilitate positioning of radiation shield 106 and/or imparting radiation shield 106 with a desired shape or curvature (e.g., a portion of the flexible crossbar may be bent such that the radiation shield is positioned in a curved configuration). Alternatively, or additionally, the user-selected position of radiation shield 106 may be adjusted prior to attachment of radiation shield 106 to neck 104 or base 102. In an example embodiment, radiation shield 106 may be positioned to extend entirely or nearly entirely between patient 5 and/or mattress 15/operating table 20. For example, top edge 136 of radiation shield 106 may be located at or above a lower edge of radiation source 25, and/or bottom edge 138 of radiation shield 106 may be proximate (e.g., in contact with) surgical draping, patient 5, mattress 15, and/or table 20.

Example radiation shielding device 100 may be stabilized at least partially by the weight of an object, such as a patient undergoing a medical operation. For example, base 102 may be sufficiently flat and/or positionable underneath patient 5 between a mattress 15 and a table 20 (e.g., an operating table) when patient 5 is resting in a supine position prior to or during a medical operation in which radiation source 25 is used. In some examples, base 102 can be positioned directly underneath patient 5 (e.g., between patient 5 and mattress 15) when patient 5 is resting in a supine position. The body weight of patient 5, mattress 15, and/or other objects may help stabilize and maintain the radiation shielding device 100 in an upright position.

Base 102 provides sufficient mechanical strength and stiffness to support neck 104 and radiation shield 106 during a medical operation, and/or while neck 104 and radiation shield 106 are manipulated into a desired position, such as during a set-up procedure. Base 102 may include one or more polymer materials having sufficient rigidity to at least partially support radiation shield 106. In some embodiments, base 102 may include a relatively soft or flexible material and a relatively rigid structure or frame.

Base 102 may be constructed to facilitate positioning on or between selected objects, while facilitating comfort of a patient 5 (e.g., that may be positioned, directly or indirectly, on top of base 102). In some example embodiments, base 102 has an upper surface 103 that is relatively smooth and/or free of rigid edges. When base 102 is positioned directly underneath patient 5, an upper surface 103 promotes physical comfort of patient 5. In some embodiments, an upper surface 103 is cushioned or has a gel covering. Base 102 may be flexible to easily conform to the various contours of the body of patient 5. In some example embodiments, base 102 has a lower surface that is a non-skid surface. When base 102 is positioned on a surface (e.g., table 20), unwanted movement or slipping of base 102 is prevented by frictional interaction between the non-skid surface of base 102 and the surface. For example, the lower surface of base 102 may include a relatively soft or rubberized material.

Radiation shielding device 100 is configured to facilitate effective imaging of a target area of the patient while providing radiation protection to a healthcare practitioner and/or the patient. In some examples, the radiation shielding device can provide a level of attenuation of at least 50%, 60%, 75%, 80%, or 95% at a radiation level of about 90 kVp (peak kilovoltage), such as directed according to ASTM Test Method F3094-14. For example, the radiation shielding device can provide a level of attenuation ranging between 50% and 75%, between 75% and 90%, between 90% and 95%, or between 50% to 99% at a radiation level of about 90 kVp. In some examples, the radiation shielding device can provide a level of attenuation of at least 50%, 60%, 75%, 80%, or 95% at a radiation level of about 70 kVp (peak kilovoltage). For example, the radiation shielding device can provide a level of attenuation ranging between 50% and 75%, between 75% and 90%, between 90% and 95%, or between 50% to 99% at a radiation level of about 70 kVp. In some examples, the radiation shielding device can provide a level of attenuation of at least 50%, 60%, 75%, 80%, or 95% at a radiation level of about 105 kVp (peak kilovoltage). For example, the radiation shielding device can provide a level of attenuation ranging between 50% and 75%, between 75% and 90%, between 90% and 95%, or between 50% to 99% at a radiation level of about 70 kVp.

In some example embodiments, base 102 is substantially radio-transparent such that passage of radiation 10 generated by radiation source 25 is not inhibited. A radio-transparent base may thus facilitate effective imaging of a target area of the patient and/or facilitate the use of relatively lower levels of radiation. In some examples, base 102 is made from one or more radio-transparent materials such as a radio-transparent polymer, fabric, non-woven, etc.

Alternatively or in addition, at least portions of base 102 include a radiation-blocking material that inhibits the passage of radiation 10 generated by radiation source 25 (e.g., such that high levels of radiation on a first side may be reduced to levels safe for a healthcare practitioner on the second side). For example, a portion of base 102 positionable under patient 5 (e.g., a portion opposite attachment location 112) may be substantially radio-transparent, and another portion (e.g., proximate to attachment location 112) may be substantially radio-opaque to provide radiation shielding for the healthcare practitioner. In some example embodiments, base 102 may have an opening, cut-out region, or other area that permits transmission of radiation 10. For example, base 102 may be “U”-shaped with each leg of the “U” extending under the object (e.g., patient 5) and the central area of the “U” permitting radiation 10 to pass therethrough while not disrupting or distorting the imaging. In various example embodiments, radiation-blocking materials of base 102 may include one or more of barium, tin, aluminum, tungsten, lead, and/or other radiation-attenuating metals.

In some example embodiments, base 102 may be adhesively attached to an object (e.g., patient 5, mattress 15, table 20, or radiation equipment). Adhesive attachment may facilitate stability of radiation shielding device 100 in maintaining radiation shield 106 in a selected position. In some examples, base 102 includes an adhesive on at least a portion of a lower surface of base 102. Attaching base 102 to an object may include removing an adhesive release liner to expose an adhesive layer and contacting the adhesive layer with the object to adhere the base to the object. Base 102 may be stabilized in an upright position by adhesive attachment with the object, without placement beneath the weight of patient 5. Alternatively, or additionally, a base having an adhesive layer on a lower surface for attachment of the base to an object may have a relatively small footprint.

In some example embodiments, base 102 includes an attachment location 112. Neck 104 extends between first end 105a and second end 105b proximate attachment location 112. Neck 104 facilitates at least partially supporting radiation shield 106 at a location that is not directly above attachment location 112. Radiation shield 106 may be suspended at least partially over the patient (e.g., away from a perimeter edge of the operating table or mattress).

In some example embodiments, base 102 includes an attachment location 112 for neck 104. Attachment location 112 (e.g., including one or more magnets, adhesives, fasteners, etc.) facilitates secure attachment and/or maintains second end 105b of neck 104 in a fixed location relative to base 102, radiation source 25, and/or target location of patient 5 (e.g., during a medical operation). In some example embodiments, neck 104 is an elongate, flexible component configured to at least partially support radiation shield 106 at a location spaced from base 102 and/or attachment location 112 between neck 104 and base 102. Neck 104 may include a gooseneck tube, a flexible arm, or a flexible pipe, for example. In various example embodiments, neck 104 may include a pliable material such as a soft metal or a soft plastic combined with a high-strength metal (e.g., steel or stainless steel). For example, neck 104 may include a high-strength metal inner spring coil that provides neck 104 with the necessary stiffness to retain a position and a soft outer covering (e.g., a soft metal, silicone, or polyethylene) that provides neck 104 with flexibility and pliability to be adjusted to a user-selected position. In some embodiments, neck 104 may include an elongate, rigid neck, and/or include portions having a pre-defined shape that are not readily deformable by a healthcare practitioner during use.

Neck 104 may be independently positioned by the user (e.g., a healthcare practitioner) relative to base 102, radiation source 25, and/or patient 5. Neck 104 may be positioned at an angle relative to base 102. For example, radiation shielding device 100 defines an angle (a) between the surface of base 102 and a straight line extending between first end 105a and second end 105b (e.g., a lower portion 107b of neck 104). Angle α may be selected by the healthcare practitioner considering, for example, the medical procedure being performed, the medical tools being used, the location of the procedure, the anatomy of the patient, etc. Angle α may be between about 360° and 180°, 180° and 135°, 135° and 45°, 105° and 75°, 45° and 0°, or about 90°. For example, neck 104 may be oriented substantially vertically (e.g., vertically or otherwise within 15° of the direction of the gravitational force or the Z-axis) and base 102 may be oriented substantially horizontally (e.g., horizontally or otherwise within 15° of a direction perpendicular to the gravitational force or the Z-axis). In some embodiments, angle α is an overall relative orientation of a lower portion 107b of neck 104, while an upper portion 107a of neck 104 may have varying relative angles.

In some embodiments, neck 104 may be partially or entirely flexible and may be configured to be flexed (e.g., bent in any direction). Neck 104 may be bendable along one or more portions of its length between first end 105a and second end 105b. For example, neck 104 may be adjusted to have an “S” configuration. The “S” configuration may help the user adjust shield 106 to obtain a precise placement with respect to radiation source 25 and patient 5. For example, the “S” configuration of neck 104 may facilitate positioning and/or angling of a first portion of radiation shield 106 (e.g., a portion proximate to upper portion 107a of neck 104) at a different position and/or angle with respect to a second portion of radiation shield 106 (e.g., a portion proximate to lower portion 107b of neck 104). In this manner, the “S” configuration may allow a user to position and/or angle a first portion of radiation shield 106 closer to radiation source 25 while simultaneously positioning and/or angling a second portion of radiation shield 106 away from patient 5 or vice-versa. In some examples, neck 104 may include one or more bendable portions and one or more stiff, non-bendable portions. The combination of one or more bendable portions and one or more stiff, non-bendable portions may provide the user with various non-linear configurations that can promote ergonomic positioning and enhanced radiation blocking performance. For example, the user (e.g., medical practitioner) may be able to work at a close (e.g., within inches), yet safe distance from the patient by having precise control of the placement of radiation shield 106. Neck 104 thus facilitates at least partially supporting radiation shield 106 at a location that is not directly above attachment location 112 between base 102 and neck 104. Radiation shield 106 may be suspended at least partially over the patient (e.g., away from a perimeter edge of the operating table or mattress).

Neck 104 may provide an attachment location 109 (FIG. 2A) for radiation shield 106, such as an attachment location at or proximate to first end 105a of neck 104. In some embodiments, first end 105a may include attachment location 109. In some embodiments, attachment location 109 may be in or near upper portion 107a of neck 104. In some embodiments, attachment location 109 may include a fastener, ball and socket joint, or snap-fit connector, etc. to facilitate connection of neck 104 with radiation shield 106.

In an example embodiment, radiation shield 106 is removably attachable with neck 104. A removable attachment may facilitate independent sterilization of radiation shield 106 and neck 104, and independent replacement of one or more components. Alternatively, or in addition, a removable connection between radiation shield 106 and neck 104 may provide a modular system in which components having one or more different characteristics may be used interchangeably. For example, a healthcare practitioner may select a radiation shield 106 from a set or kit of radiation shields that have one or more different dimensions, densities, radiation shielding capabilities, materials, shapes, curvatures, flexibilities, etc. The same or similar base 102 and neck 104 may thus be used to support multiple different radiation shields (e.g., radiation shields 106, 316, 424, 706) that differ in one or more characteristics.

Radiation shielding device 100 includes a support structure that supports radiation shield 106 and/or facilitates positioning in a selected position. In some embodiments, the support structure includes crossbar 108 and neck 104. Crossbar 108 may extend from first end 105a of neck 104 (e.g., in a direction transverse relative to neck 104). The radiation shield 106 may be attachable to crossbar 108 to at least partially support radiation shield 106 along a width w of radiation shield 106. In some example embodiments, crossbar 108 may be indirectly attached with neck 104. For example, neck 104 may be attached with radiation shield 106, while crossbar 108 is positioned near top edge 136 to provide structure and malleability to top edge 136 of radiation shield 106.

In some embodiments, neck 104 is attachable to radiation shield 106 at a location below a top edge 136 and bottom edge 138 such that a top portion of radiation shield 106 extends above neck 104. The support structure can include a frame, such as one or more malleable components, an internal “T”-shaped frame, etc. that supports at least the top portion (e.g., a portion proximate to top edge 136) or all of radiation shield 106. The frame may be integrally connected to radiation shield 106. In some embodiments, neck 104 attaches to radiation shield 106 at a midpoint between top edge 136 and bottom edge 138.

Crossbar 108 may support radiation shield 106 and/or facilitate a desired position/orientation of radiation shield 106. In an example embodiment, crossbar 108 may be attached to neck 104 via a fastener, ball and socket joint, or snap-fit connector, etc. Such attachments may allow articulation of crossbar 108 relative to neck 104, facilitating manipulation of radiation shield 106 into a desired position/orientation. In some embodiments, crossbar 108 may be fixedly attached to neck 104, and/or integrally or permanently attached with neck 104. In an example embodiment, crossbar can be positioned substantially perpendicular to neck 104 (e.g., exactly perpendicular or within 15° of exactly perpendicular).

In an example embodiment, crossbar 108 is flexible such that crossbar 108 may be bent to impart a desired shape or curve to radiation shield 106. Crossbar 108 may include a pliable material such as a soft metal or a soft plastic combined with a high-strength metal (e.g., steel or stainless steel). In some embodiments, crossbar 108 is a rigid crossbar that retains radiation shield 106 in a straight configuration.

A portion of radiation shield 106 proximate to top edge 136 may be attached to crossbar 108. Alternatively, or in addition, crossbar 108 may attach to any surface area of radiation shield 106 between top edge 136 and bottom edge 138. Crossbar 108 may span at least a portion of the width of radiation shield 106, extending between first side edge 140 and second side edge 142. Such a configuration may facilitate support of radiation shield 106 in a desired position/orientation, and/or may facilitate use of a relatively flexible/non-shape-stable shield material. In some embodiments, crossbar 108 may attach to any surface area of radiation shield 106 via a hook and loop fastener, an adhesive, a clamp, or a hook. In some embodiments, crossbar 108 may include a clamp that is configured to secure top edge 136 of radiation shield 106. Alternatively or additionally, radiation shield 106 includes one or more holes or perforations (e.g., proximate to top edge 136) configured to receive a hook, protrusion, or other complementary feature of crossbar 108.

During use and in an assembled configuration, crossbar 108 is positionable to be oriented substantially parallel to a side (e.g., an arm) of patient 5 or a length of table 20 or mattress 15, (e.g., exactly parallel or otherwise within 15° of a direction parallel to the patient, table, mattress, etc.). Crossbar 108 may be oriented substantially horizontally (e.g., exactly horizontally or otherwise within 15° of a direction perpendicular to the gravitational force or the Y-axis) next to a side (e.g., an arm) or surface of patient 5. In some configurations, the base may extend a greater distance (e.g., horizontally) from attachment location 112 than first end 105a of neck 104, crossbar 108, and/or radiation shield 106. For example, base 102 may extend a greater distance inward relative to the edge of the mattress or table than neck 104, crossbar 108, and/or radiation shield 106. In some examples, crossbar 108 is positionable to be oriented substantially parallel to base width w_{b} (e.g., parallel or otherwise within 15° of a direction parallel to base width w_{b}), positionable to be oriented substantially orthogonal to base length l_{b} (e.g., orthogonal or otherwise within 15° of a direction orthogonal or perpendicular to base length l_{b}), and/or positionable to be substantially angled with respect to a vertical plane that is aligned with edge 113.

In some example embodiments, crossbar 108 may be removably attached to radiation shield 106. In an example embodiment, radiation shield 106 includes a loop or opening (e.g., formed by portions of the shield material folded over itself. The loop or opening forms a passage that at least a portion of the crossbar may be positioned in. Radiation shield 106 may hang from crossbar 108 while providing continuous protection below top edge 136. In various example embodiments, crossbar 108 may be attached to radiation shield 106 via hook-and-loop fasteners, press-fit fasteners, snap fit fasteners, rivets, loops, buttons, buttonholes, slots, adhesives (e.g., adhesive landing zones), etc. In some examples, crossbar 108 may be fixedly attached to and/or integrally attached to neck 104. For example, radiation shield 106 may be folded over and crossbar may be positioned and/or fixed through the folded over material.

Radiation shield 106 may be a flexible or non-shape-stable radiation shield (e.g., such that radiation shield may fold, deform, or bend under the force of gravity). For example, radiation shield 106 may be a radiation shielding drape or fabric. Radiation shield 106 may include one or more layers of radiation shielding material, such as lead or other heavy metal, covered by a polymer, fabric, or non-metallic outer layer. In various example embodiments, radiation shield 106 may include one or more of barium, tin, aluminum, tungsten, lead, other attenuating metal, etc. Alternatively, or additionally, radiation shield 106 may include a polymeric material or a fabric material infused or interwoven with one or more materials that sufficiently block radiation to provide a zone of safe radiation levels, such as barium, tin, aluminum, tungsten, lead, or other attenuating material.

In some embodiments, radiation shield 106 includes one or more layers of radiation shielding material, such as a sheet of lead or other heavy metal. The radiation shielding material may be laminated or otherwise positioned between outer fabric, plastic, or metal layers. Alternatively, or additionally, radiation shield 106 may include a polymeric material infused with one or more materials that sufficiently block radiation to provide a zone of safe radiation levels, such as barium, tin, aluminum, tungsten, lead, other attenuating metal, etc.

The radiation shielding materials of the radiation shield 106 thus provide sufficient radiation blocking to provide a zone of safe radiation levels. A relatively high level of radiation on a first side of radiation shield 106 may be reduced to a safe level of radiation on a second side of radiation shield 106. Radiation shield 106 may hang from crossbar 108 and may maintain radiation shield 106 in an unfolded or expanded state. In some embodiments, the radiation shield 106 may include one, two, three, four, or more sheets of radiation-blocking material. For example, a radiation shield 106 including one sheet of radiation-blocking material can have a thickness equivalence to 0.10 mm of lead. In some embodiments, a radiation shield 106 including two sheets of radiation-blocking material can have a thickness equivalence to 0.15 mm of lead. In some embodiments, a radiation shield 106 including three sheets of radiation-blocking material can have a thickness equivalence to 0.20 mm of lead. In some embodiments, a radiation shield 106 including four sheets of radiation-blocking material can have a thickness equivalence to 0.25 mm of lead.

Radiation shielding device 100 may be received by a healthcare practitioner as a sterile kit, such that one or more components of radiation shielding device 100 can be removed from sterile packaging and positioned for use within the medical environment. In some example embodiments, a radiation shielding device kit may include multiple disposable bases 102 and necks 104 that can be used with a single, sterilizable, radiation shield 106 (e.g., non-shape-stable or shape-stable radiation shields), and/or vice versa. In some embodiments, a user may sterilize base 102, neck 104, and radiation shield 106 separately. In some embodiments, neck 104 and/or radiation shield 106 may be sterilized and reused independent of a base 102, or vice versa.

Referring now to FIG. 2A, base 102 may have a generally rectangular shape having a base width w_{b} and a base length l_{b}. In some embodiments, base length l_{b} is greater than base width w_{b}. For example, base length l_{b} may be about 1.5, 2, or 3 times greater than base width w_{b}. In some embodiments, base width w_{b} is greater than base length l_{b}. In some embodiments, base 102 may have a circular, polygonal, or any other suitable shape. Base width w_{b} may range between about 5 centimeters (cm) to about 50 cm, about 15 cm to about 30 cm, or about 10 cm to about 20 cm. Base length l_{b} may range between about 10 centimeters (cm) to about 60 cm, about 15 cm to about 50 cm, or about 20 cm to about 30 cm.

Radiation shield 106 may have a generally rectangular shape having a width w and a length l. In some embodiments, length l is greater than width w. For example, width w may be about 1.5, 2, or 3 times greater than length l. In some embodiments, radiation shield 106 may have a circular, polygonal, or any other suitable shape. Width w may range between about 20 cm to about 70 cm, about 25 cm to about 60 cm, or about 30 cm to about 40 cm. Length l may range between about 20 cm to about 60 cm, about 25 cm to about 50 cm, or about 30 cm to about 40 cm. A thickness of radiation shield 106 may range between about 0.1 millimeters (mm) to about 5 mm, about 0.2 to about 1 mm, or about 0.25 mm to about 0.5 mm.

Radiation shielding device 100 may have a vertical height h (e.g. a vertical distance between first end 105a and second end 105b), and neck 104 may have a total length lₙ. Length l of radiation shield 106 may be relatively great compared to height h. In some embodiments, length l of radiation shield 106 may be more than about 50%, 60%, 70%, 80%, or 90% of height h. In some embodiments, length l of radiation shield 106 may be more than 100% of height h. For example, length l of radiation shield 106 may be between 100% and 500%, between 125% and 400%, or between 150% and 250% of height h. Alternatively, or in addition, length l of radiation shield 106 may extend more than about 50%, 60%, 70%, 80%, or 90% of total length lₙ of neck 104. In some embodiments, length l of radiation shield 106 may be more than 100% of total length lₙ of neck 104. For example, length l of radiation shield 106 may be between 100% and 200%, between 100% and 150%, or between 100% and 125% of total length lₙ of neck 104. Such relative dimensions between radiation shield 106 and neck 104 or other components of radiation shielding device 100 facilitates a shielding zone that extends entirely between a bottom edge of radiation source 25 and a patient, and that may provide additional shield material that can rest on a patient to provide additional shielding (e.g., in a generally horizontal direction). Width w of radiation shield 106 may be oriented substantially parallel to base width w_{b} (e.g., parallel or otherwise within 15° of a direction parallel to base width w_{b}). Radiation shield 106 may extend from crossbar 108 in a vertical plane that is aligned with or parallel to edge 113 or base width w_{b}. Radiation shield 106 may extend from crossbar 108 in a vertical plane that is transverse to base length l_{b}. Radiation shield 106 may extend from crossbar 108 in a vertical plane that is parallel to a sagittal plane or a parasagittal of patient 5. Radiation shield 106 may extend from crossbar 108 in a vertical plane that is perpendicular to a transverse plane of patient 5.

Referring now to FIG. 2B, attachment location 112 of base 102 may be positioned near a side edge and be configured to engage second end 105b of neck 104. In some examples, second end 105b and attachment location 112 include complementary mating features, such as complementary magnets configured to magnetically secure neck 104 (e.g., second end 105b of neck 104) in a fixed position relative to base 102. In some embodiments, the magnets are sufficiently strong to enable a magnetic coupling between base 102 and neck 104 with the thickness of one or more surgical drapes 110 in between. For example, the magnets may have a sufficiently high pulling force (e.g., the amount of force one has to exert to pull on a magnet to move it away from an object, such as a steel surface or another magnet) and high magnetic field strength such that base 102 and neck 104 can remain in a fixed and upright position even if an object (e.g., one or more surgical drapes 110) is positioned between attachment location 112 and second end 105b. In some embodiments, attachment location 112 and second end 105b each include a Grade N42 or Grade N52 magnet. In some embodiments, attachment location 112 and second end 105b each include a magnet having a magnetic field strength ranging from about 33 MegaGauss-Oersteds (MGOe) to 52 MGOe. The strong magnetic coupling between base 102 and neck 104 may help maintain or improve sterility within the sterile field at the medical location by allowing the healthcare practitioner to adjust a position of the radiation shielding device 100 on a surface (e.g., a hospital bed or hospital equipment surface) without the need to move one or more surgical drapes 110.

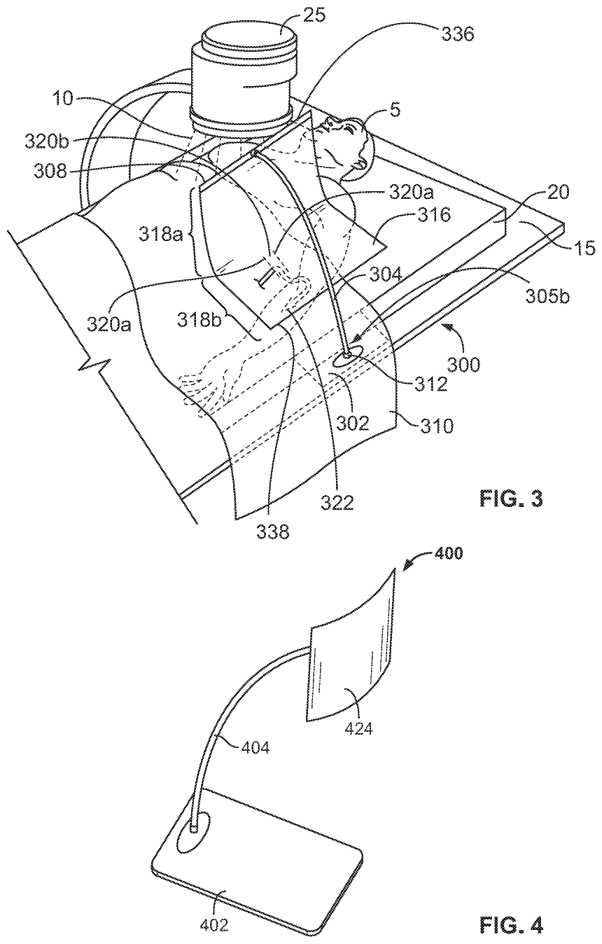

Referring now to FIG. 3, an example radiation shielding device 300 is shown, including a base 302, a neck 304, and a radiation shield 316. In various example embodiments, radiation shielding device 300 may include one or more features as described herein with reference to radiation shielding device 100. Radiation shielding device 300 may be substantially similar in construction and function in several aspects to radiation shielding device 100 discussed above. In an example embodiment, radiation shielding device 300 includes a radiation shield 316 configured to provide radiation shielding in multiple directions.

Radiation shield 316 may be a substantially flexible or non-shape-stable radiation shield, such as a flexible radiation shielding drape that may at least partially conform to a surface or object (e.g., patient 5). In an example embodiment, radiation shield 316 may include an upper portion 318a and a lower portion 318b. Upper portion 318a may hang from or otherwise be supported by neck 304 and/or crossbar 308, and lower portion 318b may be at least partially supported by an object below radiation shield 316, such as patient 5. Radiation shield 316 may thus be positioned in a folded configuration in which a fold is located at least partially between upper and lower portions 318a, 318b. Lower portion 318b is oriented at least partially transverse to upper portion 318a. In an example embodiment, lower portion 318b may be supported on top of a surface of a patient such that it may provide a generally horizontal barrier protecting the healthcare practitioner from relatively high levels of radiation (e.g., scatter radiation) during a medical operation.

In some embodiments, upper portion 318a may be supported by neck 304 extending between an object below patient 5 (e.g., table, mattress, rail, etc.) to a location above patient 5, and lower portion 318b may be at least partially supported by patient 5. Lower portion 318b may thus be supported by a first object (e.g., patient 5) located above a second object (e.g., table, mattress, rail, etc.), that supports base 302 and/or neck 304. In some embodiments, the second object may indirectly support lower portion 318b.

Radiation shield 316 may include one or more openings 320a (e.g., slits, separations, slots) through the thickness of radiation shield 316. The slits or openings 320a may facilitate passage of a medical device 322 (e.g., a catheter) from one side of radiation shield 316 to another side of radiation shield 316. For example, a portion of material 320b between first and second openings 320a may be folded or popped outwardly such that medical device 322 (e.g., a catheter) may pass through radiation shielding device 300. The portion of material 320b may be pushed toward medical device 322, for example, to substantially close an opening generated by openings 320a. The portion of material 320b may contact medical device 322 so that little or no gap is present between medical device 322, portion of material 320b, and/or other material of radiation shielding device 300. In various embodiments, such a configuration facilitates passage of medical device 322 through radiation shielding device 300 while promoting a consistent and uninterrupted zone of protection for the healthcare practitioner and/or patient 5. In various exemplary embodiments, openings 320a, may be present along about 10% of the total width of upper and/or lower portions 318a, 318b. For example, openings 320a, may be present between about 5% to 50%, 10% to 15%, or 20% to 30% of the total width of upper and/or lower portions 318a, 318b. Such dimensions may facilitate the passage of a variety and/or size of medical devices while providing adequate shielding.

In some example embodiments, radiation shield 316 may include a slit, separation, slot, etc., such as a slit, at which upper portion 318a is separated from other portions of radiation shield 316 (e.g., such as lower portion 318b). The slit may provide additional mobility of upper portion 318a independent of lower portion 318b, or vice versa, for example. A healthcare may position upper portion 318a, such as by rotating upper portion 318a, manipulating top edge 336, etc., while being less constrained (e.g., as compared to if the slit were not present). In this way, radiation shield 316 may provide additional flexibility for a healthcare practitioner to manipulate radiation shield 316 into a selected position.

In an example embodiment, upper portion 318a may extend upwardly above a surface of a patient such that it may provide a generally vertical barrier protecting the healthcare practitioner from relatively high levels of radiation (e.g., direct radiation from radiation source 25) during a medical operation. Radiation shield 316 may be manipulated via neck 304 and crossbar 308 to move upper and lower portions 318a, 318b into selected positions. Upper and lower portions 318a, 318b may define an angle relative to one another dependent on positioning of neck 304, crossbar 308, and the object (e.g., patient 5) that at least partially supports lower portion 318b. In various example embodiments, the angle may be between 45° and 180°, about 60° and 165° or between about 70° and 135°. In an example embodiment, lower portion 318b at least partially conforms to patient 5 and/or other object that at least partially supports lower portion 318b such that an angle between upper and lower portions 318a, 318b may vary at different locations of radiation shield 316.

In an example embodiment, radiation shield 316 may be manipulated by a healthcare practitioner into a configuration including two or more portions angled relative to one another. For example, radiation shield 316 may be folded such that upper portion 318a of radiation shield 316 is in a substantially vertical orientation and lower portion 318b of radiation shield 316 is in a substantially horizontal orientation. Upper portion 318a and lower portion 318b may thus form an angle between about 135° and 45°, 105° and 75°, or about 90°. In some embodiments, the angle is an overall relative orientation of upper and lower portions 318a, 318b, while the upper and/or lower portions 318a, 318b may have non-planar portions or discrete surfaces of varying relative orientations.

Alternatively, or additionally, radiation shield 316 may be manipulated to impart a selected curvature. For example, a top edge 336 may exhibit a curvature to at least partially surround a radiation field, target area or a patient, etc. One or more portions may be manipulated to impart a complex curvature such that radiation shield 316 is curved about multiple axes. A healthcare practitioner may thus manipulate radiation shield 316 into a selected configuration based on one or more of the healthcare practitioner's preferences, the medical procedure being performed, the medical tools being used, the location of the procedure, the anatomy of the patient, etc. A radiation shield 316 having upper and lower portions 318a, 318b angled relative to one another, such as in substantially horizontal and substantially vertical orientations, may provide a relatively large zone of protection from direct and/or scatter radiation.

In an example embodiment, upper and lower portions 318a, 318b, are integrally formed as a unitary component. One or more layers of radiation shield 316 may be present in both upper and lower portions 318a, 318b. The upper and lower portions 318a, 318b may be uninterrupted by separations, gaps, openings, etc., across at least a portion of a width of radiation shield 316. In this way, radiation shield 316 may promote a consistent zone of protection for a healthcare practitioner.

In some embodiments, radiation shield 316 may be operable in a fully extended configuration in which radiation shield 316 may be manipulated via neck 304 and crossbar 308 to define an angle of about 180° between upper portion 318a and lower portion 318b. That is, radiation shield 316, including upper portion 318a and lower portion 318b, may vertically extend from crossbar 308 when in an extended configuration.

In various example embodiments, the size of radiation shield 316 facilitates positioning such that radiation shield 316 (e.g., upper portion 318a) extends an entire distance between radiation source 25 and patient 5, mattress 15, and table 20, while also providing a portion (e.g., lower portion 318b) at least partially supported by patient 5, mattress 15, table 20 etc. Length l of radiation shield between top and bottom edges 336, 338, may be relatively large compared to a total length of neck 304, for example. In various example embodiments, length l of radiation shield 316 may extend more than about 50%, 60%, 70%, 80%, or 90% of total length lₙ of neck 304. In some embodiments, length l of radiation shield 316 may be more than 100% of total length lₙ of neck 304. For example, length l of radiation shield 316 may be between 100% and 200%, between 100% and 150%, or between 100% and 125% of total length lₙ of neck 304. Such relative dimensions between radiation shield 316 and neck 304 or other components of radiation shielding device 100 facilitates a shielding zone that extends entirely between a bottom edge of radiation source 25 and a patient, and that may provide additional shield material that can rest on a patient to provide additional shielding (e.g., in a generally horizontal direction).

Radiation shield 316 may be manipulated by a healthcare practitioner in the operating environment to select relative sizes of upper and lower portions. For example, the upper portion may be relatively small in a configuration in which a distance between the radiation source 25 and patient 5 is small, and more material of radiation shield 316 forms lower portion 318b. Likewise, upper portion 318a may be relatively large in a configuration in which a distance between radiation source 25 and patient 5 is large, and less material of radiation shield 316 forms lower portion 318b. In various example embodiments, radiation shield 316 may be positioned via neck 304 and/or crossbar 308 such that upper portion 318a may have a length that is about equal to the length of lower portion 318b. In some embodiments, radiation shield 316 may be positioned such that lower portion 318b has a length that is about 1.5, 2, or 3 times as long as upper portion 318a. In some embodiments, radiation shield 316 may be positioned such that upper portion 318a has a length that is about 1.5, 2, or 3 times as long as lower portion 318b.

Attachment location 312 of base 302 may be positioned near a side edge and be configured to engage second end 305b of neck 104. In some examples, second end 305b and attachment location 312 include complementary mating features, such as complementary magnets configured to magnetically secure neck 304 (e.g., second end 305b of neck 304) in a fixed position relative to base 302. In some embodiments, the magnets are sufficiently strong to enable a magnetic coupling between base 302 and neck 304 with the thickness of one or more surgical drapes 310 in between. For example, the magnets may have a sufficiently high pulling force (e.g., the amount of force one has to exert to pull on a magnet to move it away from an object, such as a steel surface or another magnet) and high magnetic field strength such that base 302 and neck 304 can remain in a fixed and upright position even if an object (e.g., one or more surgical drapes 310) is positioned in between attachment location 312 and second end 305b. In some embodiments, attachment location 312 and second end 305b each include a Grade N42 or Grade N52 magnet. In some embodiments, attachment location 312 and second end 305b each include a magnet having a magnetic field strength ranging from about 33 MegaGauss-Oersteds (MGOe) to 52 MGOe. The strong magnetic coupling between base 302 and neck 304 may help maintain or improve sterility within the sterile field at the medical location by allowing the healthcare practitioner to adjust a position of the radiation shielding device 300 on a surface (e.g., a hospital bed or hospital equipment surface) without the need to move one or more surgical drapes 310 and consequently, disrupt the sterile field. In some embodiments, the one or more surgical drapes 310, positioned in between second end 305b and attachment location 312, may have a thickness of about 0.001 inches (in.) to about 0.1 in., about 0.01 in. to about, 0.05 in., or about 0.015 in. to about 0.03 in.

Referring now to FIG. 4, an example radiation shielding device 400 is shown, including a base 402, a neck 404, and a radiation shield 424. In various example embodiments, radiation shielding device 400 may include one or more features as described herein with reference to radiation shielding devices 100 and 300. Radiation shielding device 400 may be substantially similar in construction and function in several aspects to radiation shielding devices 100 and 300 discussed above. In an example embodiment, radiation shielding device 400 includes a rigid or shape-stable radiation shield 424.

Radiation shield 424 is a substantially rigid or shape-stable shield (e.g., that maintains a predefined shape under the force of gravity) and may be attachable to neck 404 to support radiation shield 424 in a desired position/orientation. In an example embodiment, radiation shield 424 is attachable to neck 404 at a central location of radiation shield 424, such as a location slightly above a center of gravity of radiation shield 424. Such a connection location can promote the stability of radiation shield 424 and neck 404 and facilitate manipulation into a selected position/orientation by the healthcare practitioner. In various example embodiments, radiation shield 424 is attached to neck 404 via a pivotable attachment mechanism, such as a pivotable fastener, ball and socket joint, or snap-fit connector, etc. A pivotable attachment mechanism may facilitate additional flexibility for the healthcare practitioner in positioning/orienting radiation shield 424.

In an example embodiment, radiation shield 424 is removably attachable with neck 404. A removable attachment may facilitate independent sterilization of radiation shield 424 and neck 404, and independent replacement of one or more components. Alternatively, or in addition, a removable connection between radiation shield 424 and neck 404 may provide a modular system in which components having one or more different characteristics may be used interchangeably. For example, a healthcare practitioner may select a radiation shield 424 from a set or kit of radiation shields that have one or more different dimensions, densities, radiation shielding capabilities, materials, shapes, curvatures, etc. The same or similar base 402 and neck 404 may thus be used to support multiple different radiation shields 424 that differ in one or more characteristics.

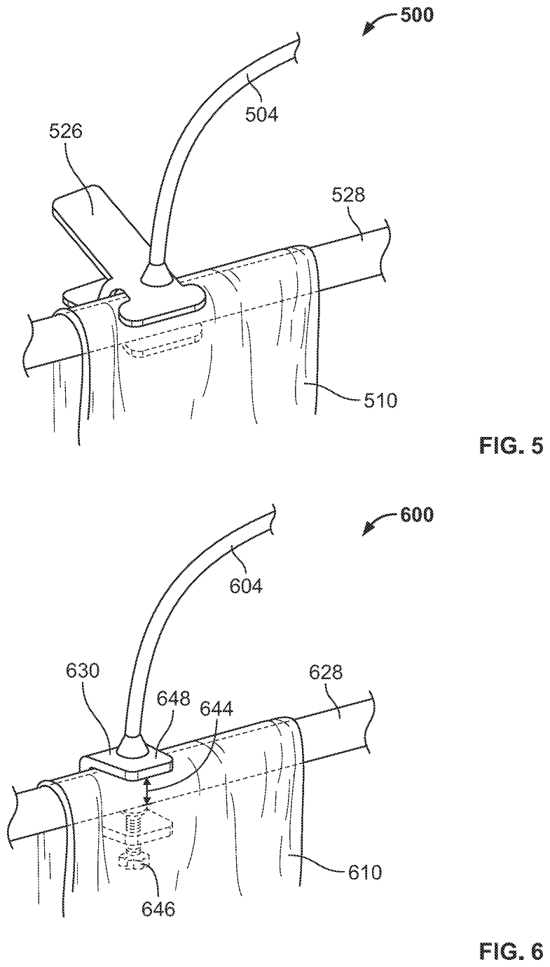

Referring now to FIG. 5, a partial view of example radiation shielding device 500 is shown. In various example embodiments, radiation shielding device 500 may include one or more features as described herein with reference to radiation shielding devices 100, 300, 400. Radiation shielding device 500 may be substantially similar in construction and function in several aspects to radiation shielding devices 100, 300, 400. In an example embodiment, radiation shielding device 500 include a clamp attachable to an object to support radiation shielding device 500 via the object.

For example, radiation shielding device 500 includes a pinch clamp 526. Neck 504 may be attachable with pinch clamp 526. A user (e.g., a healthcare practitioner) may attach radiation shielding device 500 to a surface (e.g., bed rail 528 or an edge of a table) by securing pinch clamp 526 onto the surface. In some embodiments, pinch clamp 526 opens sufficiently wide to enable the healthcare practitioner to clamp neck 504 to bed rail 528 with the thickness of one or more surgical drapes 510 in between. For example, pinch clamp 526 stabilizes radiation shielding device 500 such that neck 504 can remain in a fixed and outwardly extending position.

In an example embodiment, pinch claim 526 facilitates secure attachment of radiation shielding device 500 without altering or breaking a sterile field provided by surgical drape 510. For example, pinch claim 526 may engage a surface, such as bed rail 528, over one or more surgical drapes 510 such that the one or more surgical drapes 510 remain positioned between pinch clamp 526 and bed rail 528. Radiation shielding device 500 including pinch clamp 526 may thus help maintain or improve sterility within the sterile field at the medical location by allowing the healthcare practitioner to support radiation shielding device 500 (such that its position/orientation may be adjusted) while maintaining the positioning of one or more surgical drapes 510.

Referring now to FIG. 6, a partial view of example radiation shielding device 600 is shown. In various example embodiments, radiation shielding device 600 may include one or more features as described herein with reference to radiation shielding devices 100, 300, 400, 500. Radiation shielding device 600 may be substantially similar in construction and function in several aspects to radiation shielding devices 100, 300, 400, 500. In an example embodiment, radiation shielding device 600 include a clamp attachable to an object to support radiation shielding device 600 via the object.

For example, radiation shielding device 600 includes a screw clamp 630. Screw clamp 630 may include a screw 646 and plate 648. Neck 604 may be attachable with screw clamp 630. A user (e.g., a healthcare practitioner) may attach radiation shielding device 600 to a surface (e.g., bed rail 628 or an edge of a table) by securing screw clamp 630 onto the surface. In some embodiments, screw clamp 630 opens sufficiently wide such that opening 644 (e.g., the distance between screw 646 and plate 648) enables the healthcare practitioner to clamp neck 604 to bed rail 628 with the thickness of one or more surgical drapes 610 in between. For example, screw clamp 630 stabilizes radiation shielding device 600 such that neck 604 can remain in a fixed and outwardly extending position.

In an example embodiment, screw clamp 630 facilitates secure attachment of radiation shielding device 600 without altering or breaking a sterile field provided by surgical drape 610. For example, screw clamp 630 may engage a surface, such as bed rail 628, over one or more surgical drapes 610 such that the one or more surgical drapes 610 remain positioned between screw clamp 630 and bed rail 628. Radiation shielding device 600 including screw clamp 630 may thus help maintain or improve sterility within the sterile field at the medical location by allowing the healthcare practitioner to support radiation shielding device 600 (such that its position/orientation may be adjusted) while maintaining the positioning of one or more surgical drapes 610.

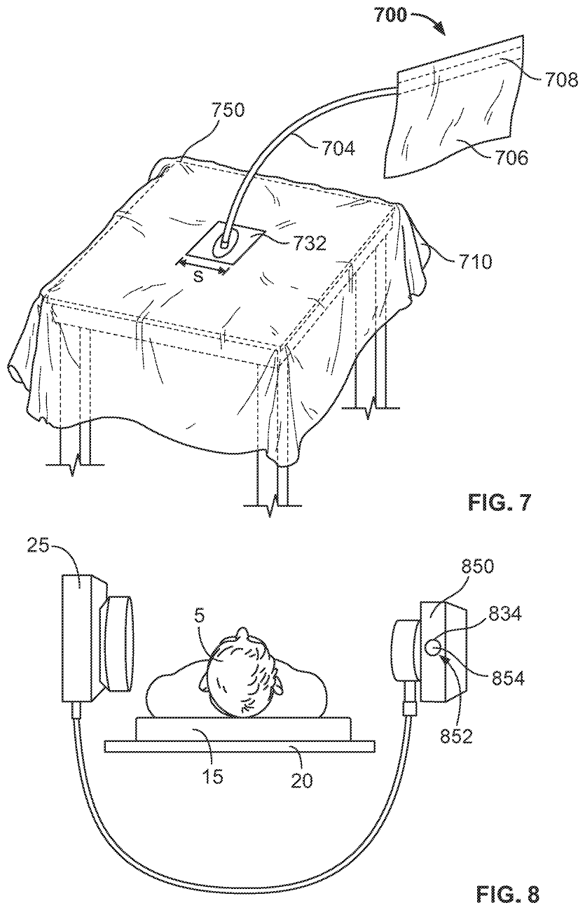

Referring now to FIG. 7, an example radiation shielding device 700 is shown, including a base 732, a neck 704, a crossbar 708, and a radiation shield 706. In various example embodiments, radiation shielding device 700 may include one or more features as described herein with reference to radiation shielding devices 100, 300, 400, 500, 600.

In an example embodiment, base 732 may be a magnetic base configured to magnetically attach radiation shielding device 700 with an object. Base 732 may be composed of a magnetic material such that at least a portion of the surface area of base 732 may be configured to magnetically attach to surface 750 (e.g. an equipment surface having a magnetic surface). Alternatively, or additionally, radiation shielding device 700 may include one or more discrete magnetic components or layers within base 732 and/or attached to a bottom surface of base 732, such that the magnets may be configured to magnetically attach to surface 750 (e.g. an equipment surface having a magnetic surface).

Base 732 may provide a relatively strong magnetic attachment and have a relatively small footprint. For example, an area of the bottom surface of base 732 (e.g., that faces the object that base 732 is attached to) may be less than 50%, less than 25%, less than 10%, less than 5%, less than 2%, or less than a surface area of a major surface of radiation shield 706. In some embodiments, a size of base 732 may be similar or slightly larger than a size of neck 704. In various example embodiments, the area of the bottom surface of base 732 may be between 100% and 1000%, 150% and 500%, or about 300% of a cross-sectional area of neck 704.

In an example embodiment, base 732 has a generally square shape having a side length s In some embodiments, side length s may be relatively small, at least in part because the magnetic attachment promotes stability of radiation shielding device 700 while have a relatively small size In various example embodiments, base 732 may have a circular, polygonal, butterfly, irregular, and/or other shape.

In an example embodiment, base 732 facilitates secure attachment of radiation shielding device 700 without altering or breaking a sterile field provided by surgical drape 710. For example, base 732 may engage surface 750 over one or more surgical drapes 710 such that the one or more surgical drapes 710 remain positioned between base 732 and surface 750.

In some example embodiments, radiation shielding devices described herein may utilize an attachment component that attaches to one or more objects and that facilitates removable attachment with a base or neck of the radiation shielding device. Referring to FIG. 8, a side view of an example radiation source 25 and clinical set-up of patient 5 is shown. In some example embodiments, the radiation shielding devices described herein may be attached to an object via an attachment component 834 configured to be attached to surface 850. For example, attachment component 834 may include a disk having a first side 852 configured to attach to an object and a second side 854 configured to attach to a radiation shielding device/neck. In an example embodiment, first side 852 of attachment component 834 includes an adhesive configured to adhere the attachment component to a surface, such as radiation source 25, operating table, or other surface in the operating environment. The adhesive surface may be located opposite second side 854 of attachment component 834. Alternatively, or in addition, first side 852 of the attachment component 834 may include a magnetic feature configured to magnetically attach to a magnetic surface of radiation source 25, operating table, or other magnetic surface in the operating environment. In some embodiments, attachment component 834 includes a Grade N42 or Grade N52 magnet, and/or a magnet having a magnetic field strength ranging from about 33 MGOe to 52 MGOe.

Second side 854 may be configured to engage with the neck or other component of the radiation shielding device, and may include a magnetic attachment, adhesive attachment, mechanical attachment, such as a snap-fit, fastener, clamp coupling, ball-and-socket coupling, etc. In some embodiments, attachment component 834 may be used to couple bases 102, 302, 402, 732, and/or necks 104, 304, 404, 704, for example. In operation, the user (e.g., a healthcare practitioner) may attach the attachment component 834 to a surface (e.g., surface 850) and attach bases 102, 302, 402, or 732 so that the radiation shielding device can be secured in a fixed and upright position, and in some embodiments provide a secure attachment through one or more surgical drapes, for example.

Attachment component 834 may provide a relatively strong magnetic, adhesive, and/or mechanical attachment and have a relatively small footprint. For example, an area of first side 852 and second side 854 may be less than 50%, less than 25%, less than 10%, less than 5%, less than 2%, or less than a surface area of a major surface of radiation shields 106, 316, 424, 706. In some embodiments, a size of first side 852 and second side 854 may be similar or slightly larger than a size of necks 104, 304, 404, 504, 604, 704. In various example embodiments, the areas of first side 852 and second side 854 may be between 100% and 1000%, 150% and 500%, or about 300% of a cross-sectional area of necks 104, 304, 404, 504, 604, 704. In various example embodiments, attachment component 834 may have a cylindrical, circular, rectangular, square, polygonal, butterfly, irregular, and/or other shape.

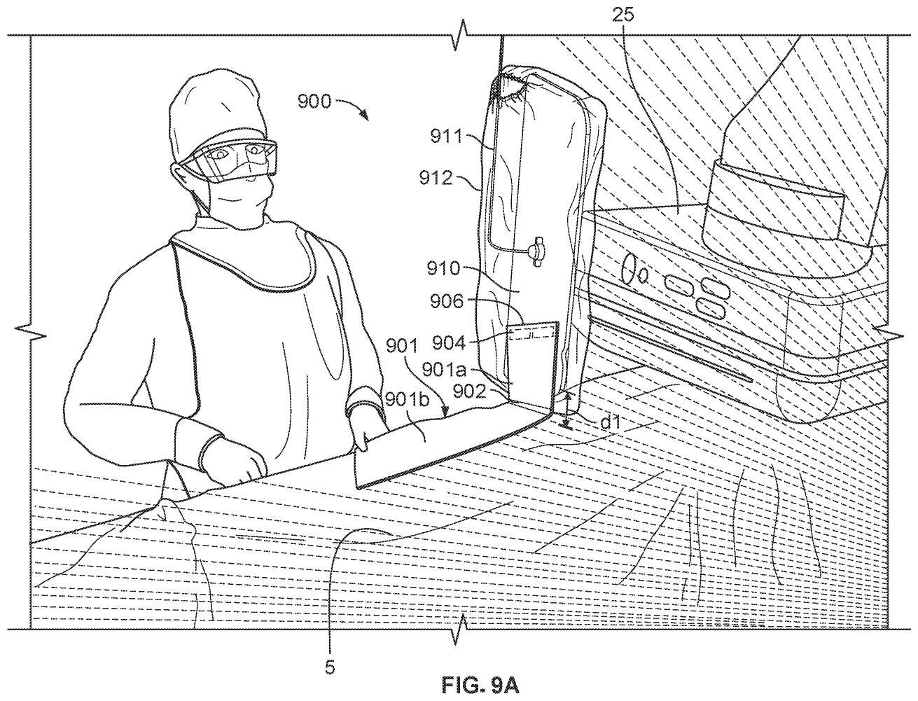

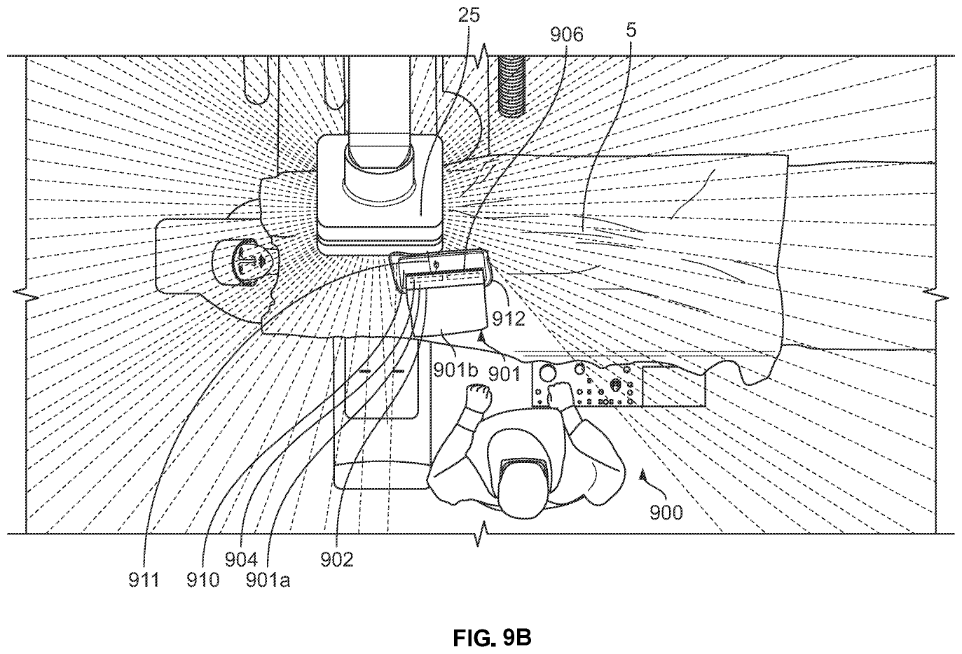

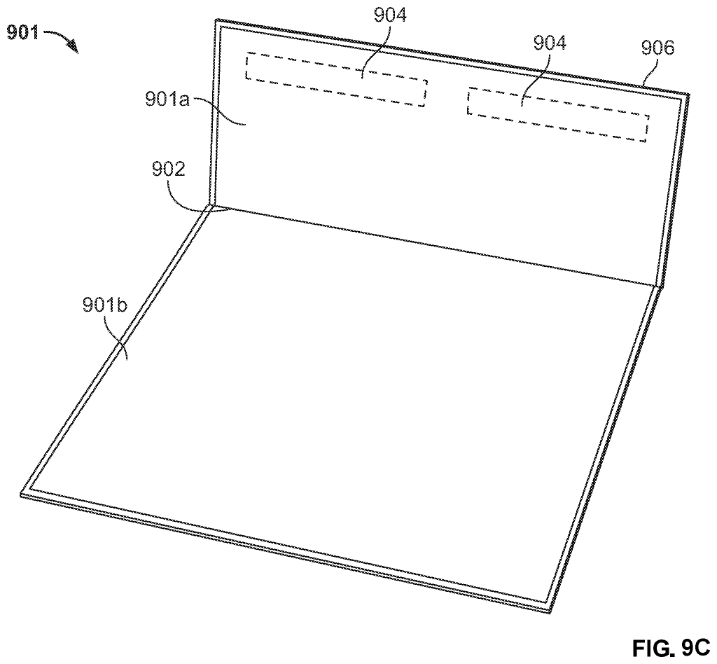

Referring now to FIGS. 9A-9C, an example radiation shielding system 900 is shown. In various example embodiments, radiation shielding system 900 includes one or more features as described herein with reference to radiation shielding devices 100, 300, 400, 500, 600, 700 and FIGS. 1-8. In an example embodiment, radiation shielding system 900 includes a radiation shield 901 arranged having a first portion 901a in a first orientation and a second portion 901b in a second orientation different than the first orientation.

In an example embodiment, radiation shield 901 is a substantially flexible or non-shape-stable radiation shield, such as a flexible radiation shielding drape that may at least partially conform to a surface or object (e.g., patient 5). In an example embodiment, radiation shield 901 includes an upper portion 901a and a lower portion 901b. Upper portion 901a is positionable to hang from or otherwise be supported by another object, such as an object in an operating environment. Lower portion 901b may be at least partially supported by an object below radiation shield 901, such as patient 5. Radiation shield 901 may thus be positioned in a folded configuration in which a fold 902 is located at least partially between upper and lower portions 901a, 901b. Lower portion 901b is oriented at least partially transverse to upper portion 901a. In an example embodiment, lower portion 901b may be supported on top of a surface of a patient 5 such that lower portion 901b provides a generally horizontal barrier protecting the healthcare practitioner from relatively high levels of radiation (e.g., scatter radiation) during a medical operation.

In an example embodiment, upper and lower portions 901a, and 901b are continuous portions of radiation shield 901. For example, radiation shield 901 is substantially flexible or non-shape stable such that it can be selectively flexed or folded to define a location of fold 902. Alternatively or additionally, radiation shield 901 can include one or more predetermined fold locations at which the radiation shield may be folded by a user to arrange upper portion 901a at a transverse orientation relative to lower portion 901b. The fold locations (e.g., fold 902) may be provided by a weld (e.g., sonic weld), reduced thickness, increased flexibility, and/or combinations thereof, for example. In some embodiments, a consistently flexible radiation shield 901 may provide a fold location of fold 902.

In some embodiments, one or more portions of radiation shield 901 are relatively rigid and/or shape-stable. For example, upper portion 901a and lower portion 901b may each be relatively ridge and/or shape-stable and separated by a flexible fold location (e.g., fold 902) such that the upper and lower portions 901a and 901b may be selectively oriented relative to one another.

In some example embodiments, radiation shield 901 may include a slit, separation, slot, etc., such as a slit, at which upper portion 901a is at least partially separated or separable from other portions of radiation shield 901 (e.g., such as lower portion 901b). The slit may provide additional mobility of upper portion 901a independent of lower portion 901b, or vice versa, for example, or the slit may provide a pathway through which a medical device (e.g., a cannula) may be passed through the shield 901. In an example embodiment, the slit is located proximate fold 902, such as directly adjacent or collinear with an axis of fold 902 (e.g. about which the portions are folded relative to one another). A healthcare professional may position upper portion 901a, such as by rotating upper portion 901a, manipulating top edge 906, etc., while being less constrained (e.g., as compared to if the slit were not present). In this way, radiation shield 901 may provide additional flexibility for a healthcare practitioner to manipulate radiation shield 901 into a selected position.

In an example embodiment, radiation shield 901 is manipulated by a healthcare practitioner in the operating environment into a configuration including two or more portions angled relative to one another. For example, radiation shield 901 is folded such that upper portion 901a is in a substantially vertical orientation and lower portion 901b is in a substantially horizontal orientation. Upper portion 901a and lower portion 901b may thus form an angle between about 135° and 45°, 105° and 75°, or about 90°. In some embodiments, the angle is an overall relative orientation of upper and lower portions 901a, 901b, while the upper and/or lower portions 901a, 901b may have non-planar portions or discrete surfaces of varying relative orientations.

Alternatively, or additionally, radiation shield 901 may be manipulated to impart a selected curvature. For example, a top edge 906 may exhibit a curvature to at least partially conform to an adjacent structure, surround a radiation field, target area or a patient, etc. One or more portions may be manipulated to impart a complex curvature such that radiation shield 901 is curved about multiple axes. A healthcare practitioner may thus manipulate radiation shield 901 into a selected configuration based on one or more of the healthcare practitioner's preferences, the medical procedure being performed, the medical tools being used, the location of the procedure, the anatomy of the patient, etc. A radiation shield 901 having upper and lower portions 901a, 901b angled relative to one another, such as in substantially horizontal and substantially vertical orientations, may provide a relatively large zone of protection from direct and/or scatter radiation.

In an example embodiment, upper and lower portions 901a, 901b, are integrally formed as a unitary component. One or more layers are present in both upper and lower portions 901a, 901b. The upper and lower portions 901a, 901b may be uninterrupted by separations, gaps, openings, etc., across at least a portion of a width of radiation shield 901. In this way, radiation shield 901 may promote a consistent zone of protection for a healthcare practitioner.

Radiation shield 901 is, in some embodiments, attachable to a structure in the operating environment to at least partially support radiation shield 901. For example, radiation shield 901 includes one or more attachment features configured to secure radiation shield 901 (e.g., upper portion 901a of radiation shield 901) in a fixed position/orientation relative to the patient 5, radiation component 25, and/or the structure radiation shield 901 is attached to.

In an example embodiment, radiation shielding system 900 includes a rigid radiation shield 910 positionable adjacent patient 5 and/or radiation system 25. For example, the rigid radiation shield 910 is maintained in the operating environment for repeated use with radiation system 25. In an example embodiment, the rigid radiation shield 910 is mounted or suspended from the ceiling, wall, etc., of the operating environment and includes a transparent radiation shield. Before use, the rigid radiation shield 910 is, in an example embodiment, covered by a sterile cover 912. The rigid radiation shield 910 may be raised/lowered/rotated, etc., such as via a support arm 911, into a selected position relative to patient 5, radiation component 25, healthcare practitioner, etc. In an example embodiment, the rigid radiation shield is not supported by or otherwise in contact with the patient 5, operating table, and/or radiation component 25 when positioned for use to shield a healthcare practitioner from radiation during a medical procedure. For example, a gap may be present between the rigid radiation shield 910 and the patient 5, operating table, etc.

In optional embodiments in which sterile cover 912 is used, sterile cover 912 is disposed and/or sanitized after use with a patient and replaced by a new or sanitized cover for a subsequent medical procedure. For example, sterile cover 912 includes a plastic or flexible sleeve 912 that covers all or some of the rigid radiation shield 910 and support arm 911. Sterile sleeve 912 is positioned over the rigid radiation shield 910 to provide a barrier between the rigid radiation shield 910 and the operating environment. Potential contaminants are prevented from entering the operating field from the barrier (e.g., within the sleeve 912) and environmental contaminants are prevented from coming into direct contact with the barrier. In some embodiments, rigid radiation shield 910 and sleeve 912 are at least partially transparent (e.g., such that the healthcare practitioner can at least partially see objects through rigid radiation shield 910 and sleeve 912.

Flexible shield 901 can facilitate sterility in the operating environment. In an example embodiment, flexible shield 901 is sterile and maintained in a sterile package until removal in the operating environment at a time of use. For example, flexible shield 901 is packaged in a disposable sterile package. In some embodiments, flexible shield 901 can be used in conjunction with, or instead of, sterile cover 912. For example, at least a portion of flexible shield 901 covers or overlaps a surface of rigid radiation shield 910, promoting a sterile field. In some embodiments, flexible shield 901 is sized to wrap around a portion of rigid radiation shield 910, such as around a lower portion of rigid radiation shield 910, thereby promoting a sterile field.

Radiation shield 901 includes an attachment component that facilitates removable attachment with another component in an operating environment. For example, radiation shield 901 includes attachment component 904 that facilitates attachment with rigid radiation shield 910. In an example embodiment, attachment component 904 includes a magnet or magnetic component. The attachment component 904 may be located at least partially externally, such as located on an external surface of radiation shield 901 (e.g., visible to a user). Alternatively or additionally, attachment component is located at least partially internally within radiation shield 901, such as located entirely between outer layers of radiation shield 901, and or associated with upper portion 901a of radiation shield 901 (e.g., located in a fixed position proximate an edge of upper portion 901a of radiation shield 901).

The attachment component 904 removably secures radiation shield 901 with rigid radiation shield 910, such as by magnetic attraction to a complementary magnetic component. For example, the radiation shield 901 includes a first magnetic component and the complementary magnetic component of rigid radiation shield 910 includes a second magnetic component. The first magnetic component includes a magnet or a ferrous material, and the second magnetic component includes a complementary ferrous material or magnet. Alternatively or additionally, attachment component 904 includes first and second magnetic components. One or both of the first and second magnetic components are selectively positionable relative to the radiation shield 901 and/or radiation shield 910 to secure the radiation shield 901 in a desired position.

In some embodiments, attachment component 904 removably secures radiation shield 901 with rigid radiation shield 910 by magnetic engagement with a complementary magnetic component located on a near side of the rigid radiation shield 910 (e.g., opposite the radiation equipment). For example, sterile sleeve 912 is sandwiched (e.g., directly or indirectly) between attachment component 904 (including a first magnetic component) of radiation shield 901 and a second magnetic component affixed to rigid radiation shield 910. Alternatively or additionally, attachment component 904 (including the first magnetic component) removably secures radiation shield 901 with rigid radiation shield 910 via a second magnetic component located on an opposite side of the rigid radiation shield 910. The rigid radiation shield 910 and/or sleeve 912 are sandwiched (e.g., directly or indirectly) between attachment component 904 of radiation shield 901 and the second magnetic component.

In some embodiments, the second magnetic component is located within the sleeve 912 and the radiation shield 901 and attachment component 904 are located outside the sleeve 912 while the radiation shield 901 is secured relative to radiation shield 910. Such a configuration facilitates secure placement of radiation shield 901 relative to patient 5, radiation component 25, and/or rigid radiation shield 910, for example, without breaking the sleeve 912 or requiring removal of the sleeve 912. Such a configuration can thus facilitate maintenance of sterility of the operating environment and enhance workflow of the healthcare professional.

Alternatively or additionally, attachment component 904 secures radiation shield 901 by attachment with a complementary component having one or more features of attachment component 834 (e.g., described above with reference to FIG. 8). For example, a magnetic component may include a magnetic disk having a first side configured to attach to rigid radiation shield 910. The first side includes an adhesive to attach to a surface of rigid radiation shield 910. The attachment component 904 couples radiation shield 901 relative to radiation shield 910 via magnetic engagement between attachment component 904 (e.g., including a first magnetic component) and the magnetic disc. Magnetic engagement facilitates attachment of radiation shield 901 through the sleeve 912 and/or other components covering rigid radiation shield 910. In operation, the user (e.g., a healthcare practitioner) may attach the complementary attachment component (e.g., having one or more features of attachment component 834) to a surface, such as a surface of rigid radiation shield 910, and securely position radiation shield 901 in magnetic engagement so that the radiation shield 901 can be secured in a fixed and upright position, and in some embodiments provide a secure attachment through one or more surgical drapes, for example.

In an example embodiment, radiation shielding system 900 includes magnetic components of sufficient strength to enable a magnetic coupling through the thickness of sterile sleeve 912 covering rigid radiation shield 910, and/or a thickness of rigid radiation shield 910. For example, the magnetic components may have a sufficiently high pulling force (e.g., the amount of force one has to exert to pull on a magnet to move it away from an object) and high magnetic field strength such that radiation shield 901 can remain in a fixed and upright position even if an object (e.g., a sleeve covering rigid radiation shield 910) is positioned between attachment component 904 and rigid radiation shield 910. In some embodiments, attachment component 904 includes a Grade N42 or Grade N52 magnet (e.g., first and/or second magnetic components include a Grade N42 or Grade N53 magnet). In some embodiments, attachment component 904 includes a magnet having a magnetic field strength ranging from about 33 MegaGauss-Oersteds (MGOe) to 52 MGOe. The strong magnetic coupling can help maintain or improve sterility within the sterile field at the medical location by allowing the healthcare practitioner to adjust a position of the radiation shielding device 901 on a surface (e.g., a hospital bed or hospital equipment surface) without the need to remove sleeve 912 covering rigid radiation shield 910.

In an example embodiment, attachment component 904 of radiation shield 901 includes a clamp attachable to an object to support radiation shielding device 901 via the object. For example, attachment component 904 includes a pinch clamp. A user (e.g., a healthcare practitioner) may attach radiation shield 901 to a surface, such as rigid radiation shield 910, a bed rail, an edge of a table, or radiation component 25, by securing the pinch clamp onto the surface. In some embodiments, a complementary feature associated with rigid radiation shied 910 facilitates attachment of the pinch clamp. For example, rigid radiation shield 910 includes a protuberance, ledge, or other outwardly-extending feature (e.g., attached to, or integrally formed as a unitary component, with rigid radiation shield 910). In some embodiments, the pinch clamp opens sufficiently wide to enable the healthcare practitioner to clamp radiation shield 901 to rigid radiation shied 910 with the thickness of one or more sleeves 912 in between. For example, the pinch clamp stabilizes radiation shield 901 such that upper portion 901a is secured in a generally upright or vertical position, blocking a gap between rigid radiation shield 910 and patient 25, for example.

In an example embodiment, attachment component 904 including a pinch clamp facilitates secure attachment of radiation shield 901 without altering or breaking a sterile area provided by sleeve 912. For example, the pinch clamp may engage a surface of rigid radiation shield 910 over one or more sleeves 912 such that the one or more surgical sleeves 912 remain positioned between the pinch clamp and rigid radiation shield 910. Attachment component 904 including a pinch clamp may thus help maintain or improve sterility within the sterile field at the medical location by allowing the healthcare practitioner to support radiation shield 901 in a selected orientation while maintaining the positioning of one or more sleeves 912.

In various example embodiments, attachment component 904 of radiation shield 901 includes a hook-and-loop fastener, press-fit fastener, snap fit fastener, rivet, loop, button, buttonhole, slot, adhesive, etc. In some embodiments, such fasteners can facilitate secure and user-friendly attachment, especially in scenarios in which sterile sleeve 912 is not used. The attachment component 904 include one or more such features that attaches with a complementary component of rigid radiation shield 910 and/or directly to sleeve 912. Alternatively or additionally, attachment component 904 of radiation shield 901 includes one or more holes, perforations, loops, etc. (e.g., proximate to a top edge) configured to receive a hook, protrusion, or other complementary feature of rigid radiation shield 910 and maintain upper portion 901a in an generally upright or vertical position.

In various example embodiments, the size of radiation shield 901 facilitates positioning such that radiation shield 901 (e.g., upper portion 901a) extends an entire distance between a gap between rigid radiation shield 910 and patient 5 (e.g., gap dl), while also providing a portion (e.g., lower portion 901b) at least partially supported by patient 5. In an example embodiment, radiation shield 901 may be manipulated by a healthcare practitioner in the operating environment to select relative sizes of upper and lower portions 901a, 901b. For example, the upper portion 901a may be relatively small in a configuration in which a gap (d1) between the rigid radiation shield 910 and patient 5 is small, and more material of radiation shield 901 forms lower portion 901b. Likewise, upper portion 901a may be relatively large in a configuration in which a gap (dl) between rigid radiation shield 910 and patient 5 is large, and less material of radiation shield 901 forms lower portion 901b. In some embodiments, radiation shield 901 may be positioned such that lower portion 901b has a length that is about 2, 3, 4, 5, 10, or more times as long as upper portion 901a, or vice versa.

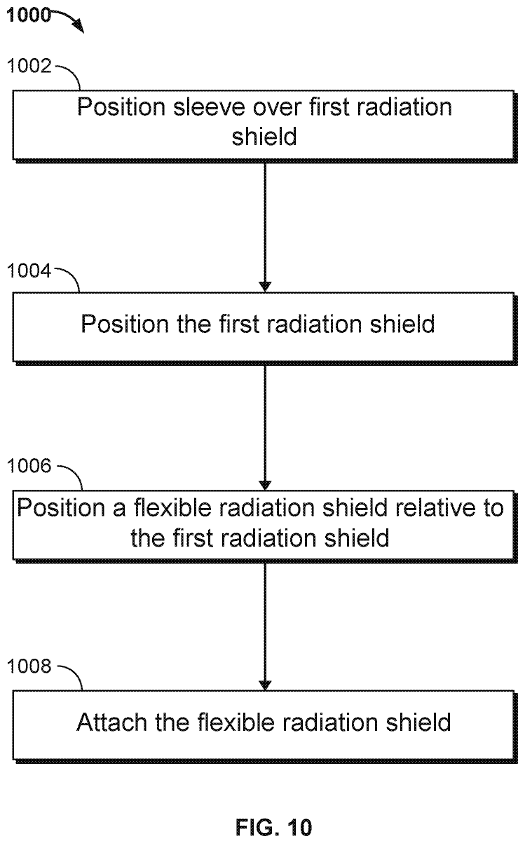

Referring now to FIG. 10, an example flow diagram is shown illustrating a method 1000 of operating a radiation shielding system to shield a healthcare practitioner from radiation during a medical procedure. In some example embodiments, method 1000 does not require a particular order of operations shown in FIG. 10 and described below, and operations may be added or eliminated.

Example method 1000 includes optional operation 1002 of positioning a sleeve, such as a sterile sleeve, over a rigid radiation shield. In an example embodiment, the rigid radiation shield is a reusable shield that is maintained in the operating environment for repeated use with a radiation system. For example, the rigid radiation shield is mounted or suspended from the ceiling, wall, etc., of the operating environment and includes a transparent radiation shield. Before use, the sleeve is positioned over the rigid radiation shield to at least partially cover the rigid radiation shield, providing a zone of sterility around the rigid radiation shield.

Example operation 1004 includes positioning the rigid radiation shield in a desired position relative to a patient, radiation component, and/or other object in the operating environment. Positioning the rigid radiation shield may include raising or lowering a height, adjusting a lateral or horizontal position, rotating the orientation, etc. In an example embodiment, positioning the rigid radiation shield results in a selected position in which the rigid radiation shield is not supported by or otherwise in contact with the patient, operating table, or a radiation component, and is positioned for use to shield a healthcare practitioner from radiation during a medical procedure. In some embodiments, a gap is present between the rigid radiation shield and the patient.

Example operation 1006 includes positioning a flexible radiation shield to at least partially cover the gap between the rigid radiation shield and the patient. In an example embodiment, positioning the flexible radiation shield includes folding or otherwise orientating a first portion of the flexible radiation shield in a first orientation and a second portion of the flexible radiation shield in a second orientation that is different and transverse to the first orientation. The first portion is oriented in a generally vertical orientation by adjusting the first portion relative to the second portion about a fold location. The second portion is oriented in a generally horizontal orientation.

Example operation 1008 includes attaching the flexible radiation shield to the rigid radiation shield (e.g., and optional sleeve assembly). The flexible radiation shield includes one or more features of radiation shield 901 described above with reference to FIGS. 9A-9C, for example, that facilitate attachment to another object in the operating environment. In an example embodiment, operation 1006 includes attaching the flexible radiation shield to be at least partially supported by the rigid radiation shield. The first (e.g., upper) portion of the flexible radiation shield is attached to the sleeve and/or rigid radiation shield via an attachment component of the flexible radiation shield (e.g., such as attachment component 904 described above). For example, the upper portion of the flexible radiation shield is at least partially supported and maintained in a generally vertical position by attachment with the rigid radiation shield and the lower portion of the flexible radiation shield is at least partially supported by a patient in a generally horizontal position.

In an example embodiment, operation 1008 includes attaching the flexible radiation shield to the rigid radiation shield while the sleeve remains between the flexible radiation shield and the rigid radiation shield. Alternatively or additionally, attaching the flexible radiation shield is completed without breaking or removing the sleeve from the rigid radiation shield. Such operation facilitates secure placement of the flexible radiation shield to cover a gap between the rigid radiation shield and patient while promoting sterility by not requiring that the sleeve be removed or broken.

While this specification contains many specific implementation details, these should not be construed as limitations on the scope of any technology or of what may be claimed, but rather as descriptions of features that may be specific to particular embodiments. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Particular embodiments of the subject matter have been described. Other embodiments are within the scope of the following claims.

What is claimed is:

1. A method of shielding radiation, comprising:
orienting a rigid radiation shield in a selected position relative to a patient, thereby forming a gap between a bottom of the rigid radiation shield and the patient; and
attaching a flexible radiation shield to the rigid radiation shield such that the flexible radiation shield includes:
a first generally vertical portion that covers the gap between the rigid radiation shield and the patient.

2. The method of claim 1, comprising covering at least a portion of the rigid radiation shield with a sterile cover.

3. The method of claim 2, wherein after attaching the flexible radiation shield to the rigid radiation shield, the sterile cover is at least partially located between the rigid radiation shield and the flexible radiation shield while the flexible radiation shield is at least partially supported by the rigid radiation shield.

4. The method of claim 1, wherein the flexible radiation shield includes a second portion oriented transverse to the first portion while the first portion is attached to the rigid radiation shield.

5. The method of claim 4, wherein the second portion is at least partially supported by the patient while the first portion is at least partially supported by the rigid radiation shield.

6. The method of claim 5, comprising positioning the flexible radiation shield relative to the patient.

7. The method of claim 6, wherein positioning the flexible radiation shield comprises folding the first portion relative to the second portion.

8. The method of claim 3, wherein attaching the flexible radiation shield comprises attaching by an attachment component of the flexible radiation shield.

9. The method of claim 8, wherein the attachment component includes a first and a second magnetic component, wherein the first magnetic component comprises a magnet and the second magnetic component comprises a ferrous material.

10. The method of claim 1, wherein the radiation shield is flexible.

11. The method of claim 10, wherein the radiation shield comprises a predetermined fold location, and positioning the flexible radiation shield comprises folding the flexible radiation shield about the predetermined fold location.

12. The method of claim 1, wherein the radiation shield comprises a radiation shielding sheet.

13. The method of claim 9, wherein the attachment component of the flexible radiation shield includes a clamp.

14. The method of claim 1, wherein the attachment component includes a first magnetic component, and attaching the flexible radiation shield includes securing the flexible radiation shield by magnetic engagement of the first magnetic component.

15. The method of claim 14, comprising adhesively securing a second magnetic component to the rigid radiation shield, and wherein attaching the flexible radiation shield comprises magnetically engaging the first magnetic component with the second magnetic component.

16. A method of shielding radiation, comprising:
orienting a rigid radiation shield in a selected position relative to a patient;
attaching a flexible radiation shield to the rigid radiation shield such that the flexible radiation shield includes a first generally vertical portion at least partially supported by the rigid radiation shield and that covers a gap between the rigid radiation shield and the patient, and a second generally horizontal portion that is at least partially supported by the patient.

17. The method of claim 16, comprising positioning the flexible radiation shield relative to the patient, wherein the radiation shield comprises a predetermined fold location and positioning the flexible radiation shield comprises folding the flexible radiation shield about the predetermined fold location.

18. A radiation shielding device, comprising:
a first flexible portion comprising a radiation shielding material;
a second flexible portion comprising a radiation shielding material;
an attachment component affixed with the first flexible portion, the attachment component configured to magnetically engage an object to secure the first flexible portion in a selected position relative to the second flexible portion.

19. The radiation shielding device of claim 18, wherein the first flexible portion is at least partially supported by attachment of the attachment component to an object in an operating environment while the second flexible portion is at least partially supported by a patient.

20. The radiation shielding device of claim 19, wherein the magnetic attachment component is configured to secure the radiation shielding device over a plastic cover without breaking or removing the plastic cover.
